(19) United States
(12) Patent Application Publication
(10) Pub. No.: US 2007/0010553 A1
(43) Pub. Date: Jan. 11, 2007

(54) New use

(75) Inventor: Anders Lehmann, Vastra Frolunda (SE)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: AstraZeneca AB, Sodertalje (SE); NPS PHARMACEUTICALS, INC., Salt Lake City, UT (US)

(21) Appl. No.: 11/471,696

(22) Filed: Jun. 21, 2006

Related U.S. Application Data
(60) Provisional application No. 60/695,417, filed on Jul. 1, 2005.

(51) Int. Cl. A61K 31/4709; A61K 31/4439

(52) U.S. Cl. 514/314; 514/341

(57) ABSTRACT

The present invention relates to the use of metabotropic glutamate receptor 5 (mGluR5) antagonists for the treatment or prevention of cough.

### FIELD OF THE INVENTION

The present invention relates to the use of metabotropic glutamate receptor 5 (mGluR5) antagonists for the treatment or prevention of cough.

### BACKGROUND OF THE INVENTION

The metabotropic glutamate receptors (mGluR) are G-protein coupled receptors that are involved in the regulation and activity of many synapses in the central nervous system (CNS). Eight metabotropic glutamate receptor subtypes have been identified and are subdivided into three groups based on sequence similarity. Group I consists of mGluR1 and mGluR5. These receptors activate phospholipase C and increase neuronal excitability. Group II, consisting of mGluR2 and mGluR3 as well as group III, consisting of mGluR4, mGluR6, mGluR7 and mGluR8 are capable of inhibiting adenylyl cyclase activity and reduce synaptic transmission. Several of the receptors also exist in various isoforms, occurring by alternative splicing (Chen, C-Y et al., Journal of Physiology (2002), 538.3, pp. 773-786; Pin, J-P et al., European Journal of Pharmacology (1999), 375, pp. 277-294; Bräuner-Osborne, H et al. Journal of Medicinal Chemistry (2000), 43, pp. 2609-2645; Schoepp, D. D, Jane D. E. Monn J A. Neuropharmacology (1999), 38, pp. 1431-1476).

The compounds of formula II below have been described in WO01/12627 A1 and WO02/068417 A2 as mGluR5 antagonists. The compounds have therein been described as being useful in the treatment of various CNS disorders such as senile dementia, schizophrenia, Alzheimer's disease and anxiety.

Cough can be caused by a variety of factors, including allergies, asthma, smoking or gastroesophageal reflux disease. Coughing may begin with irritation of nerves in the respiratory tract. The irritation may come from a plug of mucus in the airway, or from exposure to a chemical aerosol or from a number of other causes. Cough may be caused by lung disease as well as other diseases. A subject suffering from cough will often experience said cough as irritating and the coughing will often have an impact on the quality of life due to e.g. problems sleeping, thus decreasing the productivity of a subject. Current pharmacotherapy includes antitussives to help stop coughing. Some medicines are available without prescription, while others must be prescribed by a physician.

There is still a need for a cough therapy which does not have the disadvantages of influencing the respiratory rate, and which has an acceptable bioavailability. The object of the present invention was therefore to find a novel therapy for preventing or treating cough.

### OUTLINE OF THE INVENTION

It has now surprisingly been found that metabotropic glutamate receptor 5 (mGluR5) antagonists are useful for the treatment or prevention of cough.

Consequently, the present invention is directed to the use of a metabotropic glutamate receptor 5 antagonist for the manufacture of a medicament for the treatment or prevention of cough.

One embodiment of the present invention is the use of a compound of formula II 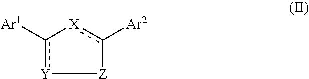
wherein X, Y, and Z are independently selected from the group consisting of N, O, S, C, and CO wherein at least one of X, Y, and Z is a heteroatom;
Ar¹ and Ar² are independently selected from the group consisting of a heterocyclic or fused heterocyclic moiety containing 1 to 4 heteroatoms selected from the group consisting of N, O, and S and an aromatic moiety selected from the group consisting of phenyl, benzyl, 1-naphthyl, 2-naphthyl, fluorenyl, anthrenyl, indenyl, phenanthrenyl, and benzonaphthenyl, wherein the Ar¹ and Ar² moieties are optionally substituted with one or more moieties selected from the group consisting of —F, —Cl, —Br, —I, —OR, —SR, —SOR, —SO₂R, —SO₂NRR′, —OCOR, —OCONRR′, —NRCOR′, —NRCO₂R′, —CN, —NO₂, —CO₂R, —CONRR′, —C(O)R, —CH(OR)R′, —CH₂(OR), —R, and -A-(CH₂)n-NRR′;
wherein R or R′ is selected from the group consisting of H, CF₃, C₁-C₁₀ alkyl, cycloalkyl, alkyl-aryl, alkyl-heteroaryl, heterocycloalkyl, aryl and where R and R′ may combine to form a ring, and A is defined as CH₂, O, NH, S, SO, SO₂ and n is 1, 2, 3, or 4; and
wherein the heterocyclic or fused heterocylic moiety preferably is selected from the group consisting of quinolyl, quinazolyl, quinoxalyl, 2-pyrimidyl, 4-pyrimidyl, 5-pyrimidyl, 2-pyridyl, 3-pyridyl, 4-pyridyl, and pyrazyl;
or a pharmaceutically acceptable salt or an optical isomer thereof, for the manufacture of a medicament for the treatment or prevention of cough.

The Ar¹ moiety is generally defined as a heterocyclic moiety, and the Ar¹ moiety is generally defined as a carbocylic moiety. Ar¹ and Ar² can be monocyclic or fused bicyclic groups. In one embodiment, Ar² is defined as an aryl or alkaryl moiety. In one embodiment, Ar¹ is defined as a heterocyclic, heteroaryl or heteroarylalkyl moiety. The ring systems encompassed by Ar¹ can contain up to four heteroatoms, independently selected from the group consisting of N, S, and O. When Ar¹ is a heteroaryl ring or ring system, it preferably contains one or two heteroatoms. At least one of the heteroatoms preferably is nitrogen (N). The heterocyclic or fused heterocylic moiety preferably is selected from the group consisting of quinolyl, quinazolyl, quinoxalyl, 2-pyrimidyl, 4-pyrimidyl, 5-pyrimidyl, 2-pyridyl, 3-pyridyl, 4-pyridyl, and pyrazyl.

Monocyclic Ar¹ groups include, but are not limited to: thiazoyl, furyl, pyranyl, 2H-pyrrolyl, thienyl, pyrroyl, imidazoyl, pyrazoyl, pyridyl, pyrazinyl, pyrimidinyl, and pyridazinyl moieties. Monocyclic Ar² group include but are not limited to phenyl and benzyl. Fused bicyclic Ar² include, but are not limited to, naphthyl, fluorenyl, anthrenyl, indenyl, phenanthrenyl, and benzonaphthenyl. Fused bicyclic Ar¹ groups include, but are not limited to: benzothiazole, benzimidazole, 3H-indolyl, indolyl, indazoyl, purinyl, quinolizinyl, isoquinolyl, quinolyl, phthalizinyl, naphthyridinyl, quinazolinyl, cinnolinyl, isothiazolyl, quinoxalinyl indolizinyl, isoindolyl, benzothienyl, benzofuranyl, isobenzofuranyl, and chromenyl moieties. In one embodiment, Ar¹ is a 2-pyridyl moiety. In one embodiment, Ar² is a substituted phenyl moiety.

The Ar¹ and Ar² moieties optionally may independently be substituted with one or more moieties selected from the group consisting of halogen, C₁-C₃ alkyl, C₁-C₃ O-alkyl, —OH, —OCF₃, —COOR, —COR, —SOR, —SO₂NRR′, —NRR′, —CN, —CF₃, —CO—NRR′, -A-(CH₂)ₙ—NRR′, wherein A is C, O, N, SO, SO₂, and R and R′ are independently selected from the group consisting of C₁-C₃ alkyl, H, cycloalkyl, heterocycloalkyl, aryl, and n is 1, 2, 3, or 4.

In one embodiment of the invention, the compound is selected from the group consisting of 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(3-methoxyphenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 2-(3-chlorophenyl)-4-(pyridin-2-yl)-1,3-oxazole, 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole and pharmaceutically acceptable salts thereof.

In a further embodiment of the invention, the compound is selected from the group consisting of 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methylphenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-(3-chlorophenyl)-4-(pyridin-2-yl)-1,3-oxazole, 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole; 2-(3-bromophenyl)-4-(pyridin-2-yl)-1,3-oxazole, 2-(3-cyanophenyl)-4-(pyridin-2-yl)-1,3-oxazole, 5-(3-hydroxyphenyl)-3-(pyridine-2-yl)-1,2-oxazole, 5-(3-cyanophenyl)-3-(pyridin-2-yl)-1,2-oxazole, 3-(2-pyridyl)-5-(3-iodophenyl)-1,2,4-triazole, 3-(2-pyridyl)-5-(5-chloro-2-aminophenyl)-1,2,4-oxadiazole and pharmaceutically acceptable salts thereof.

In a further embodiment of the invention, the compound is selected from the group consisting of 3-(2-pyridyl)-5-(3-methoxyphenyl)-1,2,4-oxadiazole, 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole, 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methyl phenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-(3-chlorophenyl)-4-(pyridin-2-yl)-1,3-oxazole, 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole and pharmaceutically acceptable salts thereof.

In still a further aspect of the invention, the compound is selected from the group consisting of 3-(5-Methyl-pyrid-2-yl)-5-(3-cyanophenyl)-1,2,4-oxadiazole, 3-(5-Cyano-pyrid-2-yl)-5-(3-cyanophenyl)-1,2,4-oxadiazole, 3-(2-Pyridyl)-5-(5-bromo-2-methoxyphenyl)-1,2,4-oxadiazole, 3-(2-Pyridyl)-5-(5-bromo-2-fluorophenyl)-1,2,4-oxadiazole, 3-(2-Pyridyl)-5-(5-cyano-2-fluorophenyl)-1,2,4-oxadiazole, 3-(2-Pyridyl)-5-(5-bromopyrid-3-yl)-1,2,4-oxadiazole, 3-(2-Pyridyl)-5-(5-chloro-pyrid-3-yl)-1,2,4-oxadiazole, 3-(5-Cyanopyrid-2-yl)-5-(5-bromo-pyrid-3-yl)-1,2,4-oxadiazole, 3-(5-Fluoropyrid-2-yl)-5-(5-bromo-pyrid-3-yl)-1,2,4-oxadiazole, 3-(2-Pyridyl)-5-(2-thiomethoxy-pyrid-3-yl)-1,2,4-oxadiazole, 3-(2-Pyridyl)-5-(5-methylpyrid-3-yl)-1,2,4-oxadiazole, 3-(2-Pyridyl)-5-(5-methoxypyrid-3-yl)-1,2,4-oxadiazole, 3-(2-Pyridyl)-5-(3-cyano-5-methylphenyl)-1,2,4-oxadiazole, 3-(2-Pyridyl)-5-(3-fluoro-5-bromophenyl)-1,2,4-oxadiazole, 3-(2-Pyridyl)-5-(3-iodo-5-bromophenyl)-1,2,4-oxadiazole, 3-(5-Fluoro-2-pyridyl)-5-(3-fluoro-5-bromophenyl)-1,2,4-oxadiazole, 3-(2-Pyridyl)-5-(3-iodo-5-(methylphenylester)-1,2,4-oxadiazole, 3-(2-Pyridyl)-5-(3-methoxy-5-(methoxycarbonyl)phenyl)-1,2,4-oxadiazole, 3-(2-Pyridyl)-5-(3-bromo-5-cyanophenyl)-1,2,4-oxadiazole, 3-(2-Pyridyl)-5-(5-cyano-3-iodophenyl)-1,2,4-oxadiazole, 3-(5-Cyano-2-pyridyl)-5-(3-bromophenyl)-1,2,4-oxadiazole, 3-(5-Cyano-2-pyridyl)-5-(3-cyano-5-fluorophenyl)-1,2,4-oxadiazole, 3-(5-Cyano-2-pyridyl)-5-(3-bromo-5-fluorophenyl)-1,2,4-oxadiazole, 3-(2-Pyridyl)-5-(5-cyano-2-methoxyphenyl)-1,2,4-oxadiazole, 3-(2-Pyridyl)-5-(2-cyano-5-methoxyphenyl)-1,2,4-oxadiazole, 3-(2-Pyridyl)-5-(5-cyano-pyrid-3-yl)-1,2,4-oxadiazole, 3-(2-Pyridyl)-5-(3-cyano-5-(methoxycarbonyl)phenyl)-1,2,4-oxadiazole, 3-(2-Pyridyl)-5-(5-phenyl-pyrid-3-yl)-1,2,4-oxadiazole, 3-(2-Pyridyl)-5-(3-cyano-5-methoxyphenyl)-1,2,4-oxadiazole, 3-(2-Pyridyl)-5-(3-cyano-5-hydroxyphenyl)-1,2,4-oxadiazole, 3-(2-Pyridyl)-5-(3-cyano-5-propoxyphenyl)-1,2,4-oxadiazole, 2-(3-Cyanophenyl)-4-(pyridin-2-yl)-1,3-thiazole, 2-((3-Bromo-5-iodophenyl)-4-pyridin-2-yl)-1,3-oxazole, 2-(2-Pyridyl)-5-(3-iodophenyl)-1,3,4-oxadiazole, 2-(2-Pyridyl)-5-(3-cyanophenyl)-1,3,4-oxadiazole, 2-(2-Pyridyl)-5-(3-cyanophenyl)-1,3,4-triazole, 3-(5-Chloropyrid-2-yl)-5-(3-cyano-5-fluorophenyl)-1,2,4-oxadiazole, 3-(5-Chloropyrid-2-yl)-5-(3-cyano-5-chlorophenyl)-1,2,4-oxadiazole, 3-(5-Chloropyrid-2-yl)-5-(3-chloro-5-fluorophenyl)-1,2,4-oxadiazole, 3-(5-Chloropyrid-2-yl)-5-(3-cyano-5-methoxyphenyl)-1,2,4-oxadiazole, 3-(5-Fluoropyrid-2-yl)-5-(3-cyano-5-chlorophenyl)-1,2,4-oxadiazole, 3-(5-Fluoropyrid-2-yl)-5-(3-fluoro-5-chlorophenyl)-1,2,4-oxadiazole, 3-(5-Fluoropyrid-2-yl)-5-(3-cyano-5-methoxyphenyl)-1,2,4-oxadiazole, 3-(5-Cyanopyrid-2-yl)-5-(3-cyano-5-chlorophenyl)-1,2,4-oxadiazole, 3-(5-Cyanopyrid-2-yl)-5-(3-fluoro-5-chlorophenyl)-1,2,4-oxadiazole, 3-(5-Cyanopyrid-2-yl)-5-(3-cyano-5-methoxyphenyl)-1,2,4-oxadiazole, 3-(5-Fluoropyrid-2-yl)-5-(3,5-di-cyanophenyl)-1,2,4-oxadiazole, 3-(3-(4-Dimethylaminobutoxy)-pyrid-2-yl)-5-(3-cyano-5-fluorophenyl)-1,2,4-oxadiazole, 3-(3-(5-Dimethylaminopentyloxy)-pyrid-2-yl)-5-(3-Cyano-5-fluorophenyl)-1,2,4-oxadiazole, 3-(3-(6-Dimethylaminohexyloxy)-pyrid-2-yl)-5-(3-cyano-5-fluorophenyl)-1,2,4-oxadiazole and pharmaceutically acceptable salts thereof.

In still a further aspect of the invention, the compound is selected from the group consisting of 3-(2-Pyridyl)-5-(3-allyloxy-5-(methoxycarbonyl)phenyl)-1,2,4-oxadiazole, 3-(2-Pyridyl)-5-(3-N,N-dimethylaminophenyl)-1,2,4-oxadiazole, 3-(2-Pyridyl)-5-(3-cyano-5-(4-pyridyl)phenyl)-1,2,4-oxadiazole, 3-(2-Pyridyl)-5-(2-methoxy-5-(4-pyridyl)phenyl-1,2,4-oxadiazole, 3-(2-pyridyl)-5-(2-fluoro-5-(4-pyridyl)phenyl)-1,2,4-oxadiazole, 3-(2-Pyridyl)-5-(3-fluoro-5-(4-pyridyl)phenyl)-1,2,4-oxadiazole, 3-(2-Pyridyl)-5-(3-fluoro-5-(3-pyridyl)phenyl)-1,2,4-oxadiazole, 3-(2-Pyridyl)-5-(2-fluoro-5-(3-pyridyl)phenyl)-1,2,4-oxediazole, 3-(2-Pyridyl)-5-(2-methoxy-5-(3-pyridyl)phenyl)-1,2,4-oxadiazole, 3-(2-Pyridyl)-5-(3-cyano-5-(3-pyridyl)phenyl)-1,2,4-oxadiazole, 3-(5-Fluoro-2-pyridyl)-5-(3-fluoro-5-(3-pyridyl)phenyl)-1,2,4-oxadiazole, 3-(2-Pyridyl)-5-(5-(3-pyridyl-pyrid-3-yl))-1,2,4-oxadiazole, 3-(5-Fluoropyrid-2-yl)]-5-[5-(3-pyridyl-pyrid-3-yl)]-3-1,2,4-oxadiazole, 3-(5-Cyanopyrid-2-yl)-5-(3-(pyrid-3-yl)phenyl)-1,2,4-oxadiazole, 3-(5-Cyanopyrid-2-yl)-5-(3-fluoro-5-(pyrid-3-yl)phenyl)-1,2,4-oxadiazole, 3-(2-Pyridyl)-5-(3-cyano-5-(2-pyridyl)phenyl)-1,2,4-oxadiazole, 3-(2-Pyridyl)-5-(2-methoxy-5-(2-pyridyl)phenyl)-1,2,4-oxediazole, 3-(2-Pyridyl)-5-(2-fluoro-5-(2-pyridyl)phenyl)-1,2,4-oxadiazole, 3-(2-Pyridyl)-5-((3-(3-fluorophenyl)-5-fluorophenyl))-1,2,4-oxadiazole, 3-(2-Pyridyl)-5-(3-cyano-5-(3-thiophene)phenyl)-1,2,4-oxadiazole, 3-(2-Pyridyl)-5-(5-(3-thienyl)-pyrid-3-yl)-1,2,4-oxadiazole, 3-(2-Pyridyl)-5-(5-3-furyl)-pyrid-3-yl)-1,2,4-oxadiazole, 3-(2-Pyridyl)-5-(5-(3-methoxyphenyl)-pyrid-3-yl)-1,2,4-oxadiazole, 3-(2-Pyridyl)-5-(3-cyano-5-(5-pyrimidyl)phenyl)-1,2,4-oxadiazole, 3-(2-Pyridyl)-5-(3-cyano-5-(3-aminophenyl)phenyl)-1,2,4-oxadiazole, 3-(2-Pyridyl)-5-(3-cyano-5-(3-fluorophenyl)phenyl)-1,2,4-oxediazole, 3-(2-Pyridyl)-5-(5-(5-pyrimidyl)-pyrid-3-yl)-1,2,4-oxadiazole, 3-(2-Pyridyl)-5-(3-aminomethyl-5-cyanophenyl)-1,2,4-oxadiazole, 3-(2-Pyridyl)-5-(5-(2-propenyl)-pyrid-3-yl)-1,2,4-oxadiazole, 3-(2-Pyridyl)-5-(3-cyano-5-vinylphenyl)-1,2,4-oxadiazole, 3-(2-Pyridyl)-5-(3-cyano-5-(2-hydroxyethyl)phenyl)-1,2,4-oxadiazole, 3-(2-Pyridyl)-5-(3-cyano-5-(2,3-dichloropropoxy)phenyl)-1,2,4-oxadiazole, 3-(2-Pyridyl)-5-(3-allyloxy-5-carboxyphenyl)-1,2,4-oxadiazole, 3-(2-Pyridyl)-5-(3-allyloxy-5-cyanophenyl)-1,2,4-oxadiazole, 3-(2-Pyridyl)-5-(5-cyano-3-(3-hydroxypropyn-1-yl)phenyl)-1,2,4-oxadiazole, 3-(2-Pyridyl)-5-(2-N-methylaminophenyl)-1,2,4-oxadiazole, and 3-(2-Pyridyl)-5-(5-(3-N-benzyl-1,2,5,6-tetrahydropyridine)-pyrid-3-yl)-1,2,4-oxadiazole.

In still a further aspect of the invention, the compound is selected from the group consisting of 3-(2-pyridyl)-5-(3-fluorophenyl)-1,2,4-oxadiazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 3-(2-pyridyl)-5-(5-chloro-2-aminophenyl)-1,2,4-oxadiazole, 5-(3-hydroxyphenyl)-3-(pyridin-2-yl)-1,2-oxazole, 5-(3-cyanophenyl)-3-(pyridin-2-yl)-1,2-oxazole, 3-(2-pyridyl)-5-(3-chlorophenyl)-1,2,4-triazole, 3-(2-pyridyl)-5-(3-iodophenyl)-1,2,4-triazole, 3-(2-pyridyl)-5-(5-hydroxypyrid-3-yl)-1,2,4-oxadiazole, 3-(5-fluoropyrid-2-yl)-5-(5-fluoro-3-(thiomethyl)phenyl)-1,2,4-oxadiazole, 5-(2-pyridyl)-3-(3-(1H-imidazol-1-yl)-5-fluorophenyl))-1,2,4-oxadiazole, 3-(2-pyridyl)-5-(3-cyano-5-trifluoromethylphenyl)-1,2,4-oxadiazole, 3-(5-fluoro-2-pyridyl)-5-(3-fluoro-5-trifluoromethylphenyl)-1,2,4-oxadiazole, 3-(5-cyanopyrid-2-yl)-5-(3-alloxy-5-cyanophenyl)-1,2,4-oxadiazole, 3-(5-fluoropyrid-2-yl)-5-(3-alloxy-5-cyanophenyl)-1,2,4-oxadiazole, 3-(5-cyanopyrid-2-yl)-5-(3-cyano-5-propoxyphenyl)-1,2,4-oxadiazole, 3-(5-fluoropyrid-2-yl)-5-(3-cyano-5-propoxyphenyl)-1,2,4-oxadiazole, 3-(2-pyridyl)-5-(3-cyano-5-nitrophenyl)-1,2,4-oxadiazole, 3-(5-fluoropyrid-2-yl)-5-(3-cyano-5-nitrophenyl)-1,2,4-oxadiazole, 3-(5-fluoropyrid-2-yl)-5-(3-cyano-5-dimethylaminophenyl)-1,2,4-oxadiazole, 3-(5-fluoropyrid-2-yl)-5-(3-cyano-5-(2-methoxyethoxy)phenyl)-1,2,4-oxadiazole, 3-(5-fluoropyrid-2-yl)-5-(3-cyano-5-(1H-imidazol-1-ylmethyl)phenyl)-1,2,4-oxadiazole, 3-(2-pyridyl)-5-(3-cyano-5-(methoxymethyl)phenyl)-1,2,4-oxadiazole, 3-(3-cyano-5-methoxyphenyl)-5-(2-pyridyl)-1,2,4-oxadiazole, 3-(3-cyano-5-methoxyphenyl)-5-(5-fluoropyrid-2-yl)-1,2,4-oxadiazole, 3-(5-fluoropyrid-2-yl)-5-(3-cyano-5-ethoxyphenyl)-1,2,4-oxadiazole, 3-(5-cyanopyrid-2-yl)-5-(3-cyano-5-ethoxyphenyl)-1,2,4-oxadiazole, 3-(5-chloropyrid-2-yl)-5-(3-allyloxy-5-cyanophenyl)-1,2,4-oxadiazole, 3-(5-chloropyrid-2-yl)-5-(3-cyano-5-propoxyphenyl)-1,2,4-oxadiazole, 3-(5-chloropyrid-2-yl)-5-(3-cyano-5-ethoxyphenyl)-1,2,4-oxadiazole, 3-(5-fluoropyrid-2-yl)-5-(3-cyano-5-hexyloxyphenyl)-1,2,4-oxadiazole, 3-(5-fluoropyrid-2-yl)-5-(3-cyano-5-(methoxymethyl)phenyl)-1,2,4-oxadiazole, 3-(5-fluoropyrid-2-yl)-5-(5-cyano-2-methoxyphenyl)-1,2,4-oxadiazole, 5-(5-cyano-2-methoxyphenyl)-3-(5-cyano-pyrid-2-yl)-1,2,4-oxadiazole, 3-(5-fluoro-pyrid-2-yl)-5-(3-bromophenyl)-1,2,4-oxadiazole, 5-(3-chloro-5-methyl-pyrid-4-yl)-3-(2-pyridyl)-1,2,4-oxadiazole, 5-(3-chloro-5-methoxy-pyrid-4-yl)-3-(2-pyridyl)-1,2,4-oxadiazole, 3-(3-cyano-5-methylphenyl)-5-(2-pyridyl)-1,2,4-oxadiazole, 3-(5-bromo-pyrid-3-yl)-5-(2-pyridyl)-1,2,4-oxadiazole, 3-(3-cyano-5-fluorophenyl)-5-(2-pyridyl)-1,2,4-oxadiazole, 3-(3-iodophenyl)-5-(2-pyridyl)-1,2,4-oxadiazole, 3-(3-cyanophenyl)-5-(2-pyridyl)-1,2,4-oxadiazole, 3-(3-cyano-5-dimethylamino-phenyl)-5-(2-pyridyl)-1,2,4-oxadiazole, 3-(3-cyano-5-methylphenyl)-5-(5-fluoro-pyrid-2-yl)-1,2,4-oxadiazole, 3-(3-cyano-5-fluorophenyl)-5-(5-fluoro-pyrid-2-yl)-1,2,4-oxadiazole, 3-(4-cyanophenyl)-3-(6-cyano-pyrid-2-yl)-1,2,4-oxadiazole, 5-(3-cyano-5-trifluoromethoxyphenyl)-3-(2-pyridyl)-1,2,4-oxadiazole, 5-(3-methoxycarbonyl-5-trifluoromethoxyphenyl)-3-(2-pyridyl)-1,2,4-oxadiazole, 5-(3-cyano-5-trifluoromethoxyphenyl)-3-(5-fluoro-pyrid-2-yl)-1,2,4-oxadiazole, 3-(5-cyano-pyrid-2-yl)-5-(3-cyano-5-trifluoromethoxyphenyl)-1,2,4-oxadiazole, 3-(3-cyano-5-dimethylaminophenyl)-5-(5-fluoro-pyrid-2-yl)-1,2,4-oxadiazole, 5-(5-chloro-pyrid-2-yl)-3-(3-cyano-5-dimethylaminophenyl)-1,2,4-oxadiazole, 5-(5-chloro-pyrid-2-yl)-3-(3-cyano-5-methoxyphenyl)-1,2,4-oxadiazole, 5-(5-chloro-pyrid-2-yl)-3-(6-cyano-4-methoxy-pyrid-2-yl)-1,2,4-oxadiazole, 5-(5-chloro-pyrid-2-yl)-3-(6-cyano-4-hydroxy-pyrid-2-yl)-1,2,4-oxadiazole, 5-(5-chloro-pyrid-2-yl)-3-(3-cyano-5-trifluoromethoxyphenyl)-1,2,4-oxadiazole, 5-(5-chloro-pyrid-2-yl)-3-(3-cyanophenyl)-1,2,4-oxadiazole, 5-(5-chloro-pyrid-2-yl)-3-(3-cyano-5-methylphenyl)-1,2,4-oxadiazole, 5-(5-chloro-pyrid-2-yl)-3-(3-cyano-5-fluorophenyl)-1,2,4-oxadiazole, 3-(3-cyano-5-trifluoromethoxyphenyl)-5-(2-pyridyl)-1,2,4-oxadiazole, 3-(3-fluoro-5-methoxyphenyl)-5-(2-pyridyl)-1,2,4-oxadiazole, 3-(3-cyanophenyl)-5-(5-fluoro-pyrid-2-yl)-1,2,4-oxadiazole, 3-(3-cyano-5-trifluoromethoxyphenyl)-5-(5-fluoro-pyrid-2-yl)-1,2,4-oxadiazole, 3-(3-fluoro-5-methoxyphenyl)-5-(5-fluoropyrid-2-yl)-1,2,4-oxadiazole, 3-(3,5-dimethoxyphenyl)-5-(5-fluoropyrid-2-yl)-1,2,4-oxadiazole, 3-(3-fluoro-5-(1H-imidazol-1-yl)phenyl)-5-(2-pyridyl)-1,2,4-oxadiazole, 3-(5-fluoro-2-pyridyl)-5-(3-fluoro-5-(3-pyridyl)phenyl)-1,2,4-oxadiazole, 3-(5-fluoro-2-pyridyl)-5-(3-bromo-5-(3-pyridyl)phenyl)-1,2,4-oxadiazole, 3-(5-fluoro-2-pyridyl)-5-(3-fluoro-5-methoxyphenyl)-1,2,4-oxadiazole, 3-(pyrid-2-yl)-5-(3-cyano-5-thiomethylphenyl)-1,2,4-oxadiazole, 3-(5-fluoro-pyrid-2-yl)-5-(3-cyano-5-thiomethylphenyl)-1,2,4-oxadiazole, 3-(pyrid-2-yl)-5-(3-fluoro-5-thiomethylphenyl)-1,2,4-oxadiazole, 3-(pyrid-2-yl)-5-(3-fluoro-5-thiomethylsulphoxidephenyl)-1,2,4-oxadiazole, 3-(pyrid-2-yl)-5-(3-fluoro-5-thioethylphenyl)-1,2,4-oxadiazole, 3-(pyrid-2-yl)-5-(5-fluoro-3-thiotertbutylphenyl)-1,2,4-oxadiazole, 3-(5-fluoropyrid-2-yl)-5-(3-cyano-5-methylphenyl)-1,2,4-oxadiazole, 3-(5-cyanopyrid-2-yl)-5-(3-cyano-5-thiomethylphenyl)-1,2,4-oxadiazole, 3-(5-fluoropyrid-2-yl)-5-(4-cyano-2-thienyl)-1,2,4-oxadiazole, 3-(2-pyridyl)-5-(3-cyano-5-trifluoromethylphenyl)-1,2,4-oxadiazole, 3-(5-fluoro-2-pyridyl)-5-(3-cyano-5-trifluoromethylphenyl)-1,2,4-oxadiazole, 3-(5-fluoro-pyrid-2-yl)-5-(3-(3-pyridyl)phenyl)-1,2,4-oxadiazole, 5-(3-methyl-5-(3-pyridyl)-pyrid-4-yl)-3-(2-pyridyl)-1,2,4-oxadiazole, 5-(3-methoxy-5-(3-pyridyl)-pyrid-4-yl)-3-(2-pyridyl)-1,2,4-oxadiazole, 5-(2-pyridyl)-3-(5-(3-pyridyl)-pyrid-3-yl)-1,2,4-oxadiazole, 5-(2-pyridyl)-3-(3-(3-pyridyl)-phenyl)-1,2,4-oxadiazole, 5-(5-fluoro-pyrid-2-yl)-3-(3-fluoro-5-(3-pyridyl)-phenyl)-1,2,4-oxadiazole, 3-(2-pyridyl)-5-(3-carboxy-5-methoxyphenyl)-1,2,4-oxadiazole, 3-(2-pyridyl)-5-(3-(carboxamido)-5-methoxyphenyl)-1,2,4-oxadiazole, 3-(2-pyridyl)-5-(3-cyano-5-(2-N,N-dimethylaminoethoxy)phenyl)-1,2,4-oxadiazole, 3-(2-pyridyl)-5-(3-cyano-5-(N,N-dimethylaminopropoxy)phenyl)-1,2,4-oxadiazole, 3-(2-pyridyl)-5-(3-cyano-5-(2-aminoethoxy)phenyl)-1,2,4-oxadiazole, 3-(2-pyridyl)-5-(3-cyano-5-(2-hydroxyethoxy)phenyl)-1,2,4-oxadiazole, 3-(2-pyridyl)-5-(3-cyano-5-isopropoxyphenyl)-1,2,4-oxadiazole, 3-(2-pyridyl)-5-(3-cyano-5-ethoxyphenyl)-1,2,4-oxadiazole, 3-(2-pyridyl)-5-(3-cyano-5-(2,2,2-trifluoroethoxy)phenyl)-1,2,4-oxadiazole, 3-(2-pyridyl)-5-(3-cyano-5-cyclopropylmethoxyphenyl)-1,2,4-oxadiazole, 3-(2-pyridyl)-5-(3-amino-5-cyanophenyl)-1,2,4-oxadiazole, 3-(5-fluoropyrid-2-yl)-5-(3-amino-5-cyanophenyl)-1,2,4-oxadiazole, 3-(2-pyridyl)-5-(3-cyano-5-(trifluoromethylsulfonyloxy)phenyl)-1,2,4-oxadiazole, 3-(2-pyridyl)-5-(3-cyano-5-(2-methoxy-2-oxoethoxy)phenyl)-1,2,4-oxadiazole, 3-(2-pyridyl)-5-(3-cyano-5-(2-tert-butoxy-2-oxoethoxy)phenyl)-1,2,4-oxadiazole, 3-(2-pyridyl)-5-(3-cyano-5-(methoxymethoxy)phenyl)-1,2,4-oxadiazole, 3-(2-pyridyl)-5-(3-cyano-5-(methoxyethoxy)phenyl)-1,2,4-oxadiazole, 3-(5-fluoropyrid-2-yl)-5-(3-cyano-5-cyclopentylaminophenyl)-1,2,4-oxadiazole, 3-(2-pyridyl)-5-(3-cyano-5-hexyloxyphenyl)-1,2,4-oxadiazole, 3-(2-pyridyl)-5-(3-cyano-5-(dimethylamino)carbonylphenyl)-1,2,4-oxadiazole, 3-(5-fluoropyrid-2-yl)-5-(3-cyano-5-ethylaminophenyl)-1,2,4-oxadiazole, 3-(5-fluoropyrid-2-yl)-5-(3-cyano-5-diethylaminophenyl)-1,2,4-oxadiazole, 3-(5-fluoro-pyrid-2-yl)-5-(3-fluoro-5-(1H-tetraazol-5-yl)-phenyl)-1,2,4-oxadiazole, 5-(3-fluoro-5-(1-methyl-1H-tetraazol-5-yl)-phenyl)-3-(5-fluoro-pyrid-2-yl)-1,2,4-oxadiazole, 3-(5-fluoro-2-pyridyl)-5-(3-(1-benzyl-1,2,5,6-tetrahydropyridin-3-yl)-5-fluorophenyl)-1,2,4-oxadiazole, 3-(5-fluoro-2-pyridyl)-5-(3-fluoro-5-(1H-imidazol-4-yl)phenyl)-1,2,4-oxadiazole, 1-(3-cyanophenyl)-4-(5-fluoro-2-pyridyl)-1H-imidazole, 3-(2-pyridyl)-5-(3-(1H-imidazol-1-yl)-5-thiomethoxyphenyl)-1,2,4-oxadiazole, 3-(3-cyano-5-(1H-imidazol-1-yl)phenyl)-5-(2-pyridyl)-1,2,4-oxadiazole, 3-(2-pyridyl)-5-(3-cyano-5-(1H-imidazol-1-yl)phenyl)-1,2,4-oxadiazole, 2-(3-iodophenyl)-4-(pyridin-2-yl)-1,3-thiazole, 3-(2-pyridyl)-2-(3-cyanophenyl)-furan, 3-(5-fluoro-2-pyridyl)-2-(3-cyanophenyl)-furan, 3-(5-chloro-2-pyridyl)-2-(3-cyanophenyl)-furan, 2-(2-pyridyl)-5-(5-fluoro-3-(1-imidazolyl)phenyl)-furan, 3-(5-(2-pyridyl)-2-furyl)-benzonitrile, 3-(5-(5-chloro-2-pyridyl)-2-furyl)-benzonitrile, 3-(5-(5-cyano-2-pyridyl)-2-furyl)-benzonitrile, 3-(5-(5-fluoro-2-pyridyl)-2-furyl)-benzonitrile, 3-fluoro-5-(5-(2-pyridyl)-2-furyl)-benzonitrile, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyano-5-fluorophenyl)-4-(2-pyridyl)-1,3-oxazole, 2-(5-allyloxy-3-cyanophenyl)-4-(5-fluoropyrid-2-yl)-1,3-oxazole, 2-(3-cyano-5-methoxyphenyl)-4-(pyrid-2-yl)-1,3-oxazole, 2-(3-cyano-5-methoxyphenyl)-4-(5-fluoropyrid-2-yl)-1,3-oxazole, 2-(3-cyano-5-n-propyloxyphenyl)-4-(5-fluoropyrid-2-yl)-1,3-oxazole, and 2-(3-cyano-5-methoxyphenyl)-4-(4-pyrid-2-yl)-5-chloro-1,3-oxazole.

The compounds of formula II above may be prepared as described in WO01/12627 A1 and WO02/068417 A2.

One further example of a compound having antagonistic affinity to metabotropic glutamate receptor 5, thereby being useful in accordance with the invention, is the compound 2-methyl-6-(phenylethynyl)-pyridine (often abbreviated MPEP). MPEP is commercially available from e.g. Tocris, or may be synthesized according to well-known procedures such as disclosed by K. Sonogashira et al. in Tetrahedron Lett. (1975), 50, 4467-4470.

A further example of a compound having antagonistic affinity to metabotropic glutamate receptor 5, thereby being useful in accordance with the invention, is the compound 3-[(2-Methyl-1,3-thiazol-4-yl)ethynyl]pyridine (often abbreviated MTEP), see Cosford N. D. P. et al. J. Med. Chem (2003), 46, 204-206.

Further compounds useful in accordance with the present invention are mGluR5 antagonists disclosed in WO99/26927, WO2004/014881, WO2004/014370, WO2004/014902, WO2006/014185, WO2005/080356, WO2005/080379, WO2005/080386, WO2005/080363, WO2005/066155, WO2005/082884, WO2005/080397, WO00/73283, WO2004/069813, U.S. Pat. No. 6,429,207, WO2005/044266, WO2005/044267, WO2005/044265, herein incorporated by reference.

For the purpose of this invention, the term “antagonist” should be understood as including full antagonists, inverse agonists, non-competitive antagonists or competitive antagonists, as well as partial antagonists, whereby a “partial antagonist” should be understood as a compound capable of partially, but not fully, in-activating the metabotropic glutamate receptor 5.

The term “prevention” referred to herein includes the inhibition of cough. Moreover, the “prevention” effect of the present invention provides for an inhibition of the cough reflex. Cough is an expulsive reflex initiated when foreign bodies like infection, noxious fumes or dust irritate the respiratory tract, i.e. the throat, larynx and airway. This results in a sudden expulsion of air that will clear the breathing passages from the substances causing the irritation. The cough reflex as such is quite complex and as yet not completely understood. A variety of peripheral sites are connected to the cough centre in the medulla, including the nose, auditory canal, nasopharynx, larynx, trachea, intrapulmonary bronchi and pleural surfaces and stimulation of the receptors in these site can also result in cough.

There are different kinds of coughs; some coughs are non-productive, also called dry, while others are productive (phlegm is brought up). A cough can also be either acute or chronic. A cough can also be spontaneous and voluntary. A subject suffering from cough will often experience said cough as irritating and the coughing will often have an impact on the quality of life due to e.g. problems sleeping, thus decreasing the productivity of a subject. The present invention is thus aiming for improving the life quality for subjects suffering from cough.

Cough, especially chronic cough, is caused by one or more of the following factors including: allergies and asthma, chronic obstructive pulmonary disease also called COPD (e.g. chronic bronchitis), upper respiratory tract infections, also called URTI (can cause a transient airway hyperresponsiveness), sinusitis leading to postnasal drip, acute viral infection, cancer (bronchogenic or esophageal), interstitial lung disease (e.g. emphysema or sarcoidosis), gastroesophageal reflux disease (GERD), bronchiectasis, chronic lung infections (e.g. tuberculosis), recurrent aspiration, pressure from an intrathoracic mass (e.g. thoracic aneurysm), irritation of cough receptors in the ear, lymphangitis carciomatosis, reactive airways dysfunction syndrome, vocal cord dysfunction, plentitis, psychogenic, angiotensin converting enzyme-induced cough, medical-related (ACE-inhibitors and beta blockers), congestive heart failure and smoking and other environmental irritants.

The wording cough as used throughout the present specification and claims includes chronic cough, acute cough as well as non-productive-cough, productive cough, spontaneous cough and voluntary cough. The term chronic cough is defined in accordance with Kardos P et al (The German Respiratory Society's Guideline for the Diagnosis and Treatment of Patients with Acute and Chronic Cough Medizinische Klinik 2004; 99(8):468-75) as a cough that lasts longer than 8 weeks. However, chronic cough can also be defined as a cough lasting longer than 3 weeks or as a cough lasting longer than 2 months. The term “acute cough” is also defined in accordance with the reference above as a cough lasting less than 8 weeks.

A further aspect of the invention is the use of a metabotropic glutamate receptor 5 antagonist for the manufacture of a medicament for the treatment or prevention of chronic cough.

Still a further aspect of the invention is a method for the treatment or prevention of cough, whereby a pharmaceutically and pharmacologically effective amount of a metabotropic glutamate receptor 5 antagonist is administered to a subject in need of such treatment or prevention.

Yet another aspect of the invention is a method for the treatment or prevention of chronic cough, whereby a pharmaceutically and pharmacologically effective amount of a metabotropic glutamate receptor 5 antagonist is administered to a subject in need of such treatment or prevention.

Pharmaceutical Formulations

For clinical use, the metabotropic glutamate receptor 5 antagonists are in accordance with the present invention suitably formulated into pharmaceutical formulations for oral administration. Also rectal, parenteral or any other route of administration may be contemplated to the skilled man in the art of formulations. Thus, the metabotropic glutamate receptor 5 antagonists are formulated with at least one pharmaceutically and pharmacologically acceptable carrier or adjuvant. The carrier may be in the form of a solid, semi-solid or liquid diluent.

In the preparation of oral pharmaceutical formulations in accordance with the invention, the metabotropic glutamate receptor 5 antagonist(s) to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of the active compound or compounds of the invention, vegetable oil, fat, or other suitable vehicle for soft gelatine capsules. Hard gelatine capsules may contain the active compound in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains the active substance in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the active compound and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of a compound of the invention in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, the metabotropic glutamate receptor 5 antagonists may be administered once or twice daily, depending on the severity of the patient's condition.

A typical daily dose of the metabotropic glutamate receptor 5 antagonists is from 0.1-100 mg per kg body weight of the subject to be treated, but this will depend on various factors such as the route of administration, the age and weight of the patient as well as of severity of the patient's condition.

Biological Evaluation

Evaluation of the Ability of Compounds to Prevent Cough and Reflex Bronchospasm Via Central and Peripheral Actions

Experimental Design

Citric acid-induced cough is evoked in conscious guinea pigs. Male Hartley guinea pigs (300-400 g) are placed in a chamber with a continuous flow of air. Positive and negative pressure changes within the chamber are used to monitor the expiratory and inspiratory phases of respiration. A nebuliser is connected in series with the air pump, allowing aerosol delivery of citric acid (0.1-1 M).

Cough is defined based on the visual appearance of the respiratory manoeuvre, the characteristic sound, and by a signature pressure trace typified by an expiratory effort that produces a positive chamber pressure 10-20 times higher than that associated with tidal breathing and proceeded by a rapid and pronounced inspiratory effort. All data is recorded digitally. The total number of coughs and the estimated concentration of citric acid evoking 1, 2 and 5 coughs are determined for each experiment.

The compound to be tested and vehicle is administered subcutaneously, 30 minutes prior to initiating the cough challenge. Compounds that exhibit antitussive effects are studied at several additional doses to determine relative potencies. Compounds that exhibit little if any antitussive activity at a low dose are studied at higher doses in an attempt to uncover antitussive effects. To assess CNS depressant effects of these compounds, their effects on respiratory rate and respiratory pattern (inspiratory and expiratory times) are assessed and compared to control.

1. A method for the prevention or treatment of cough, whereby a pharmaceutically and pharmacologically effective amount of a metabotropic glutamate receptor 5 antagonist, or a pharmaceutically acceptable salt or an optical isomer thereof, is administered to a subject in need of such prevention or treatment.

2. A method for the treatment or prevention of cough, whereby a pharmaceutically and pharmacologically effective amount of a compound of formula II 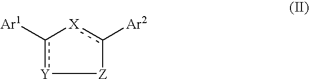
wherein X, Y, and Z are independently selected from the group consisting of N, O, S, C, and CO wherein at least one of X, Y, and Z is a heteroatom; Ar¹ and Ar² are independently selected from the group consisting of a heterocyclic or fused heterocyclic moiety containing 1 to 4 heteroatoms selected from the group consisting of N, O, and S and an aromatic moiety selected from the group consisting of phenyl, benzyl, 1-naphthyl, 2-naphthyl, fluorenyl, anthrenyl, indenyl, phenanthrenyl, and benzonaphthenyl, wherein the Ar¹ and Ar² moieties are optionally substituted with one or more moieties selected from the group consisting of —F, —Cl, —Br, —I, —OR, —SR, —SOR, —SO₂R, —SO₂NRR′, —OCOR, —OCONRR′, —NRCOR′, —NRCO₂R′, —CN, —NO₂, —CO₂R, —CONRR′, —C(O)R, —CH(OR)R′, —CH₂(OR), —R, and -A-(CH₂)—NRR′; wherein R or R′ is selected from the group consisting of H, CF₃, C₁-C₁₀ alkyl, cycloalkyl, alkyl-aryl, alkyl-heteroaryl, heterocycloalkyl, aryl and where R and R′ may combine to form a ring, and A is defined as CH₂, O, NH, S, SO, SO₂ and n is 1, 2, 3, or 4;
or a pharmaceutically acceptable salt or an optical isomer thereof, is administered to a subject in need of such treatment or prevention.

3. A method according to claim 2,
wherein, in the compound of formula II,
X and Y are N;
Z is O;
Ar¹ is 2-pyridyl and Ar² is phenyl, wherein at least one of the Ar¹ and Ar² moieties are substituted with one or more moieties selected from the group consisting of —F, —Cl, —Br, —I, SR, —SOR, —SO₂R, SO₂NRR′, —OCOR, —OCONRR′, —NRCOR′, —NRCO₂R′, —CN, —CO₂R, —CONRR′, —C(O)R, —CH(OR)R′, —CH₂(OR), and —R;
wherein R or R′ is selected from the group consisting of H, CF₃, C₁-C₁₀ alkyl, cycloalkyl, alkyl-aryl, heterocycloalkyl, aryl and where R and R′ may combine to form a ring.

4. A method according to claim 3, wherein Ar² is substituted by one or more moieties selected from the group consisting of CF₃, Cl, F, Br, CH₃, SCH₃ and CN.

5. A method according to claim 3, wherein Ar¹ is substituted by one or more moieties selected from the group consisting of CF₃, F and Cl.

6. A method according to claim 2, wherein the compound of formula II is selected from the group of compounds consisting of 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(3-methoxyphenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 2-(3-chlorophenyl)-4-(pyridin-2-yl)-1,3-oxazole and 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole and pharmaceutically acceptable salts or optical isomers thereof.

7. A method according to claim 2, wherein the compound of formula II is selected from the group of compounds consisting of 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methylphenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromediylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-[3-chlorophenyl]-4-[pyridin-2-yl]-1,3-oxazole and 2-(2,5,6-trifluorophenyl)-4-(2-pyridyl)-1,3-oxazole, 2-(3-nitrophenyl)-4-(2-pyridyl-1,3-oxazole, 2-(3-bromophenyl)-4-(2-pyridyl)-1,3-oxazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 2-[3-bromophenyl]-4-[pyridin-2-yl]-1,3-oxazole, 2-(3-cyanophenyl)-4-(pyridin-2-yl)-1,3-oxazole, 5-[3-hydroxyphenyl]-3-[pyridine-2-yl]-1,2-oxazole, 5-[3-cyanophenyl]-3-[pyridin-2-yl]-1,2-oxazole, 3-(2-pyridyl)-5-(3-iodophenyl)-1,2,4-triazole, 3-(2-pyridyl)-5-(5-chloro-2-aminophenyl)-1,2,4-oxadiazole and pharmaceutically acceptable salts or optical isomers thereof.

8. A method according to claim 2, wherein the compound of formula II is selected from the group of compounds consisting of 3-(2-pyridyl)-5-(3-methoxyphenyl)-1,2,4-oxadiazole, 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole, 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methyl phenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-[3-chlorophenyl]-4-[pyridin-2-yl]-1,3-oxazole, 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole and pharmaceutically acceptable salts or optical isomers thereof.

9. A method according to claim 2, wherein the compound of formula II is selected from the group of compounds consisting of 3-(5-Methyl-pyrid-2-yl)-5-(3-cyanophenyl)-1,2,4-oxadiazole, 3-(5-Cyano-pyrid-2-yl)-5-(3-cyanophenyl)-1,2,4-oxadiazole, 3-(2-Pyridyl)-5-(5-bromo-2-methoxyphenyl)-1,2,4-oxadiazole, 3-(2-Pyridyl)-5-(5-bromo-2-fluorophenyl)-1,2,4-oxadiazole, 3-(2-Pyridyl)-5-(5-cyano-2-fluorophenyl)-1,2,4-oxadiazole, 3-(2-Pyridyl)-5-(5-bromopyrid-3-yl)-1,2,4-oxadiazole, 3-(2-Pyridyl)-5-(5-chloro-pyrid-3-yl)-1,2,4-oxadiazole, 3-(5-Cyanopyrid-2-yl)-5-(5-bromo-pyrid-3-yl)-1,2,4-oxadiazole, 3-(5-Fluoropyrid-2-yl)-5-(5-bromo-pyrid-3-yl)-1,2,4-oxadiazole, 3-(2-Pyridyl)-5-(2-thiomethoxy-pyrid-3-yl)-1,2,4-oxadiazole, 3-(2-Pyridyl)-5-(5-methylpyrid-3-yl)-1,2,4-oxadiazole, 3-(2-Pyridyl)-5-(5-methoxypyrid-3-yl)-1,2,4-oxadiazole, 3-(2-Pyridyl)-5-(3-cyano-5-methylphenyl)-1,2,4-oxadiazole, 3-(2-Pyridyl)-5-(3-fluoro-5-bromophenyl)-1,2,4-oxadiazole, 3-(2-Pyridyl)-5-(3-iodo-5-bromophenyl)-1,2,4-oxadiazole, 3-(5-Fluoro-2-pyridyl)-5-(3-fluoro-5-bromophenyl)-1,2,4-oxadiazole, 3-(2-Pyridyl)-5-(3-iodo-5-(methylphenylester)-1,2,4-oxadiazole, 3-(2-Pyridyl)-5-(3-methoxy-5-(methoxycarbonyl)phenyl)-1,2,4-oxadiazole, 3-(2-Pyridyl)-5-(3-bromo-5-cyanophenyl)-1,2,4-oxadiazole, 3-(2-Pyridyl)-5-(5-cyano-3-iodophenyl)-1,2,4-oxadiazole, 3-(5-Cyano-2-pyridyl)-5-(3-bromophenyl)-1,2,4-oxadiazole, 3-(5-Cyano-2-pyridyl)-5-(3-cyano-5-fluorophenyl)-1,2,4-oxadiazole, 3-(5-Cyano-2-pyridyl)-5-(3-bromo-5-fluorophenyl)-1,2,4-oxadiazole, 3-(2-Pyridyl)-5-(5-cyano-2-methoxyphenyl)-1,2,4-oxadiazole, 3-(2-Pyridyl)-5-(2-cyano-5-methoxyphenyl)-1,2,4-oxadiazole, 3-(2-Pyridyl)-5-(5-cyano-pyrid-3-yl)-1,2,4-oxadiazole, 3-(2-Pyridyl)-5-(3-cyano-5-(methoxycarbonyl)phenyl)-1,2,4-oxadiazole, 3-(2-Pyridyl)-5-(5-phenyl-pyrid-3-yl)-1,2,4-oxadiazole, 3-(2-Pyridyl)-5-(3-cyano-5-methoxyphenyl)-1,2,4-oxadiazole, 3-(2-Pyridyl)-5-(3-cyano-5-hydroxyphenyl)-1,2,4-oxadiazole, 3-(2-Pyridyl)-5-(3-cyano-5-propoxyphenyl)-1,2,4-oxadiazole, 2-(3-Cyanophenyl)-4-(pyridin-2-yl)-1,3-thiazole, 2-((3-Bromo-5-iodophenyl)-4-pyridin-2-yl)-1,3-oxazole, 2-(2-Pyridyl)-5-(3-iodophenyl)-1,3,4-oxadiazole, 2-(2-Pyridyl)-5-(3-cyanophenyl)-1,3,4-oxadiazole, 2-(2-Pyridyl)-5-(3-cyanophenyl)-1,3,4-triazole, 3-(5-Chloropyrid-2-yl)-5-(3-cyano-5-fluorophenyl)-1,2,4-oxadiazole, 3-(5-Chloropyrid-2-yl)-5-(3-cyano-5-chlorophenyl)-1,2,4-oxadiazole, 3-(5-Chloropyrid-2-yl)-5-(3-chloro-5-fluorophenyl)-1,2,4-oxadiazole, 3-(5-Chloropyrid-2-yl)-5-(3-cyano-5-methoxyphenyl)-1,2,4-oxadiazole, 3-(5-Fluoropyrid-2-yl)-5-(3-cyano-5-chlorophenyl)-1,2,4-oxadiazole, 3-(5-Fluoropyrid-2-yl)-5-(3-fluoro-5-chlorophenyl)-1,2,4-oxadiazole, 3-(5-Fluoropyrid-2-yl)-5-(3-cyano-5-methoxyphenyl)-1,2,4-oxadiazole, 3-(5-Cyanopyrid-2-yl)-5-(3-cyano-5-chlorophenyl)-1,2,4-oxadiazole, 3-(5-Cyanopyrid-2-yl)-5-(3-fluoro-5-chlorophenyl)-1,2,4-oxadiazole, 3-(5-Cyanopyrid-2-yl)-5-(3-cyano-5-methoxyphenyl)-1,2,4-oxadiazole, 3-(5-Fluoropyrid-2-yl)-5-(3,5-di-cyanophenyl)-1,2,4-oxadiazole, 3-(3-(4-Dimethylaminobutoxy)-pyrid-2-yl)-5-(3-cyano-5-fluorophenyl)-1,2,4-oxadiazole, 3-(3-(5-Dimethylaminopentyloxy)-pyrid-2-yl)-5-(3-Cyano-5-fluorophenyl)-1,2,4-oxadiazole, and [3-(3-(6-Dimethylaminohexyloxy)-pyrid-2-yl)-5-(3-cyano-5-fluorophenyl)-1,2,4-oxadiazole and pharmaceutically acceptable salts or optical isomers thereof.

10. A method according to claim 2, wherein the compound of formula II is selected from the group of compounds consisting of 3-(2-Pyridyl)-5-(3-allyloxy-5-(methoxycarbonyl)phenyl)-1,2,4-oxadiazole, 3-(2-Pyridyl)-5-(3-N,N-dimethylaminophenyl)-1,2,4-oxadiazole), 3-(2-Pyridyl)-5-(3-cyano-5-(4-pyridyl)phenyl)-1,2,4-oxadiazole, 3-(2-Pyridyl)-5-[2-methoxy-5-(4-pyridyl)phenyl]-1,2,4-oxadiazole, 3-(2-pyridyl)-5-[2-fluoro-5-(4-pyridyl)phenyl]-1,2,4-oxadiazole, 3-(2-Pyridyl)-5-(3-fluoro-5-(4-pyridyl)phenyl)-1,2,4-oxadiazole, 3-(2-Pyridyl)-5-(3-fluoro-5-(3-pyridyl)phenyl)-1,2,4-oxadiazole, 3-(2-Pyridyl)-5-[2-fluoro-5-(3-pyridyl)phenyl]-1,2,4-oxediazole, 3-(2-Pyridyl)-5-[2-methoxy-5-(3-pyridyl)phenyl]-1,2,4-oxadiazole, 3-(2-Pyridyl)-5-(3-cyano-5-(3-pyridyl)phenyl)-1,2,4-oxadiazole, 3-(5-Fluoro-2-pyridyl)-5-(3-fluoro-5-(3-pyridyl)phenyl)-1,2,4-oxadiazole, 3-(2-Pyridyl)-5-[5-(3-pyridyl-pyrid-3-yl)]-]-1,2,4-oxadiazole, 3-(5-Fluoropyrid-2-yl)]-5-[5-(3-pyridyl-pyrid-3-yl)]-3-1,2,4-oxadiazole, 3-(5-Cyanopyrid-2-yl)-5-(3-(pyrid-3-yl)phenyl)-1,2,4-oxadiazole, to 3-(5-Cyanopyrid-2-yl)-5-(3-fluoro-5-(pyrid-3-yl)phenyl)-1,2,4-oxadiazole, 3-(2-Pyridyl)-5-(3-cyano-5-(2-pyridyl)phenyl)-1,2,4-oxadiazole, 3-(2-Pyridyl)-5-[2-methoxy-5-(2-pyridyl)phenyl]-1,2,4-oxediazole, 3-(2-Pyridyl)-5-[2-fluoro-5-(2-pyridyl)phenyl]-1,2,4-oxadiazole, 3-(2-Pyridyl)-5-[(3-(3-fluorophenyl)-5-fluorophenyl)]-1,2,4-oxadiazole, 3-(2-Pyridyl)-5-(3-cyano-5-(3-thiophene)phenyl)-1,2,4-oxadiazole, 3-(2-Pyridyl)-5-[5-(3-thienyl)-pyrid-3-yl]-1,2,4-oxadiazole, 3-(2-Pyridyl)-5-[5-3-furyl)-pyrid-3-yl]-1,2,4-oxadiazole, 3-(2-Pyridyl)-5-[5-(3-methoxyphenyl)-pyrid-3-yl]-1,2,4-oxadiazole, 3-(2-Pyridyl)-5-(3-cyano-5-(5-pyrimidyl)phenyl)-1,2,4-oxadiazole, 3-(2-Pyridyl)-5-(3-cyano-5-(3-aminophenyl)phenyl)-1,2,4-oxadiazole, 3-(2-Pyridyl)-5-(3-cyano-5-(3-fluorophenyl)phenyl)-1,2,4-oxediazole, 3-(2-Pyridyl)-5-[5-(5-pyrimidyl)-pyrid-3-yl]-1,2,4-oxadiazole, 3-(2-Pyridyl)-5-(3-aminomethyl-5-cyanophenyl)-1,2,4-oxadiazole, 3-(2-Pyridyl)-5-[5[(2-propenyl)-pyrid-3-yl]-1,2,4-oxediazole, 3-(2-Pyridyl)-5-(3-cyano-5-vinylphenyl)-1,2,4-oxadiazole, 3-(2-Pyridyl)-5-(3-cyano-5-(2-hydroxyethyl)phenyl)-1,2,4-oxadiazole, 3-(2-Pyridyl)-5-(3-cyano-5-(2,3-dichloropropoxy)phenyl)-1,2,4-oxadiazole, 3-(2-Pyridyl)-5-(3-allyloxy-5-carboxyphenyl)-1,2,4-oxadiazole, 3-(2-Pyridyl)-5-(3-allyloxy-5-cyanophenyl)-1,2,4-oxadiazole, 3-(2-Pyridyl)-5-(5-cyano-3-[3-hydroxypropyn-1-yl]phenyl)-1,2,4-oxadiazole, 3-(2-Pyridyl)-5-(2-N-methylaminophenyl)-1,2,4-oxadiazole, and 3-(2-Pyridyl)-5-[5-(3-N-benzyl-1,2,5,6, tetrahydropyridine)-pyrid-3-yl]-1,2,4-oxadiazole.

11. A method according to claim 2, wherein the compound of formula II is selected from the group of compounds consisting of 3-(2-pyridyl)-5-(3-fluorophenyl)-1,2,4-oxadiazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 3-(2-pyridyl)-5-(5-chloro-2-aminophenyl)-1,2,4-oxadiazole, 5-[3-hydroxyphenyl]-3-[pyridin-2-yl]-1,2-oxazole, 5-[3-cyanophenyl]-3-[pyridin-2-yl]-1,2-oxazole, 3-(2-pyridyl)-5-(3-chlorophenyl)-1,2,4-triazole, 3-(2-pyridyl)-5-(3-iodophenyl)-1,2,4-triazole, 3-(2-pyridyl)-5-(5-hydroxypyrid-3-yl)-1,2,4-oxadiazole, 3-(5-fluoropyrid-2-yl)-5-(5-fluoro-3-(thiomethyl)phenyl)-1,2,4-oxadiazole, 5-(2-pyridyl)-3-[3-(1H-imidazol-1-yl)-5-fluorophenyl)]-1,2,4-oxadiazole, 3-(2-pyridyl)-5-(3-cyano-5-trifluoromethylphenyl)-1,2,4-oxadiazole, 3-(5-fluoro-2-pyridyl)-5-(3-fluoro-5-trifluoromethylphenyl)-1,2,4-oxadiazole, 3-(5-cyanopyrid-2-yl)-5-(3-alloxy-5-cyanophenyl)-1,2,4-oxadiazole, 3-(5-fluoropyrid-2-yl)-5-(3-alloxy-5-cyanophenyl)-1,2,4-oxadiazole, 3-(5-cyanopyrid-2-yl)-5-(3-cyano-5-propoxyphenyl)-1,2,4-oxadiazole, 3-(5-fluoropyrid-2-yl)-5-(3-cyano-5-propoxyphenyl)-1,2,4-oxadiazole, 3-(2-pyridyl)-5-(3-cyano-5-nitrophenyl)-1,2,4-oxadiazole, 3-(5-fluoropyrid-2-yl)-5-(3-cyano-5-nitrophenyl)-1,2,4-oxadiazole, 3-(5-fluoropyrid-2-yl)-5-(3-cyano-5-dimethylaminophenyl)-1,2,4-oxadiazole, 3-(5-fluoropyrid-2-yl)-5-(3-cyano-5-(2-methoxyethoxy)phenyl)-1,2,4-oxadiazole, 3-(5-fluoropyrid-2-yl)-5-(3-cyano-5-(1H-imidazol-1-ylmethyl)phenyl)-1,2,4-oxadiazole, 3-(2-pyridyl)-5-(3-cyano-5-(methoxymethyl)phenyl)-1,2,4-oxadiazole, 3-(3-cyano-5-methoxyphenyl)-5-(2-pyridyl)-1,2,4-oxadiazole, 3-(3-cyano-5-methoxyphenyl)-5-(5-fluoropyrid-2-yl)-1,2,4-oxadiazole, 3-(5-fluoropyrid-2-yl)-5-(3-cyano-5-ethoxyphenyl)-1,2,4-oxadiazole, 3-(5-cyanopyrid-2-yl)-5-(3-cyano-5-ethoxyphenyl)-1,2,4-oxadiazole, 3-(5-chloropyrid-2-yl)-5-(3-allyloxy-5-cyanophenyl)-1,2,4-oxadiazole, 3-(5-chloropyrid-2-yl)-5-(3-cyano-5-propoxyphenyl)-1,2,4-oxadiazole, 3-(5-chloropyrid-2-yl)-5-(3-cyano-5-ethoxyphenyl)-1,2,4-oxadiazole, 3-(5-fluoropyrid-2-yl)-5-(3-cyano-5-hexyloxyphenyl)-1,2,4-oxadiazole, 3-(5-fluoropyrid-2-yl)-5-(3-cyano-5-(methoxymethyl)phenyl)-1,2,4-oxadiazole, 3-(5-fluoropyrid-2-yl)-5-(5-cyano-2-methoxyphenyl)-1,2,4-oxadiazole, 5-(5-cyano-2-methoxyphenyl)-3-(5-cyano-pyrid-2-yl)-1,2,4-oxadiazole, 3-(5-fluoro-pyrid-2-yl)-5-(3-bromophenyl)-1,2,4-oxadiazole, 5-(3-chloro-5-methyl-pyrid-4-yl)-3-(2-pyridyl)-1,2,4-oxadiazole, 5-(3-chloro-5-methoxy-pyrid-4-yl)-3-(2-pyridyl)-1,2,4-oxadiazole, 3-(3-cyano-5-methylphenyl)-5-(2-pyridyl)-1,2,4-oxadiazole, 3-(5-bromo-pyrid-3-yl)-5-(2-pyridyl)-1,2,4-oxadiazole, 3-(3-cyano-5-fluorophenyl)-5-(2-pyridyl)-1,2,4-oxadiazole, 3-(3-iodophenyl)-5-(2-pyridyl)-1,2,4-oxadiazole, 3-(3-cyanophenyl)-5-(2-pyridyl)-1,2,4-oxadiazole, 3-(3-cyano-5-dimethylamino-phenyl)-5-(2-pyridyl)-1,2,4-oxadiazole, 3-(3-cyano-5-methylphenyl)-5-(5-fluoro-pyrid-2-yl)-1,2,4-oxadiazole, 3-(3-cyano-5-fluorophenyl)-5-(5-fluoro-pyrid-2-yl)-1,2,4-oxadiazole, 3-(4-cyanophenyl)-3-(6-cyano-pyrid-2-yl)-1,2,4-oxadiazole, 5-(3-cyano-5-trifluoromethoxyphenyl)-3-(2-pyridyl)-1,2,4-oxadiazole, 5-(3-methoxycarbonyl-5-trifluoromethoxyphenyl)-3-(2-pyridyl)-1,2,4-oxadiazole, 5-(3-cyano-5-trifluoromethoxyphenyl)-3-(5-fluoro-pyrid-2-yl)-1,2,4-oxadiazole, 3-(5-cyano-pyrid-2-yl)-5-(3-cyano-5-trifluoromethoxyphenyl)-1,2,4-oxadiazole, 3-(3-cyano-5-dimethylaminophenyl)-5-(5-fluoro-pyrid-2-yl)-1,2,4-oxadiazole, 5-(5-chloro-pyrid-2-yl)-3-(3-cyano-5-dimethylaminophenyl)-1,2,4-oxadiazole, 5-(5-chloro-pyrid-2-yl)-3-(3-cyano-5-methoxyphenyl)-1,2,4-oxadiazole, 5-(5-chloro-pyrid-2-yl)-3-(6-cyano-4-methoxy-pyrid-2-yl)-1,2,4-oxadiazole, 5-(5-chloro-pyrid-2-yl)-3-(6-cyano-4-hydroxy-pyrid-2-yl)-1,2,4-oxadiazole, 5-(5-chloro-pyrid-2-yl)-3-(3-cyano-5-trifluoromethoxyphenyl)-1,2,4-oxadiazole, 5-(5-chloro-pyrid-2-yl)-3-(3-cyanophenyl)-1,2,4-oxadiazole, 5-(5-chloro-pyrid-2-yl)-3-(3-cyano-5-methylphenyl)-1,2,4-oxadiazole, 5-(5-chloro-pyrid-2-yl)-3-(3-cyano-5-fluorophenyl)-1,2,4-oxadiazole, 3-(3-cyano-5-trifluoromethoxyphenyl)-5-(2-pyridyl)-1,2,4-oxadiazole, 3-(3-fluoro-5-methoxyphenyl)-5-(2-pyridyl)-1,2,4-oxadiazole, 3-(3-cyanophenyl)-5-(5-fluoro-pyrid-2-yl)-1,2,4-oxadiazole, 3-(3-cyano-5-trifluoromethoxyphenyl)-5-(5-fluoro-pyrid-2-yl)-1,2,4-oxadiazole, 3-(3-fluoro-5-methoxyphenyl)-5-(5-fluoropyrid-2-yl)-1,2,4-oxadiazole, 3-(3,5-dimethoxyphenyl)-5-(5-fluoropyrid-2-yl)-1,2,4-oxadiazole, 3-[3-fluoro-5-(1H-imidazol-1-yl)phenyl]-5-(2-pyridyl)-1,2,4-oxadiazole, 3-(5-fluoro-2-pyridyl)-5-(3-fluoro-5-(3-pyridyl)phenyl)-1,2,4-oxadiazole, 3-(5-fluoro-2-pyridyl)-5-(3-bromo-5-(3-pyridyl)phenyl)-1,2,4-oxadiazole, 3-(5-fluoro-2-pyridyl)-5-(3-fluoro-5-methoxyphenyl)-1,2,4-oxadiazole, 3-(pyrid-2-yl)-5-(3-cyano-5-thiomethylphenyl)-1,2,4-oxadiazole, 3-(5-fluoro-pyrid-2-yl)-5-(3-cyano-5-thiomethylphenyl)-1,2,4-oxadiazole, 3-(pyrid-2-yl)-5-(3-fluoro-5-thiomethylphenyl)-1,2,4-oxadiazole, 3-(pyrid-2-yl)-5-(3-fluoro-5-thiomethylsulphoxidephenyl)-1,2,4-oxadiazole, 3-(pyrid-2-yl)-5-(3-fluoro-5-thioethylphenyl)-1,2,4-oxadiazole, 3-(pyrid-2-yl)-5-(5-fluoro-3-thiotertbutylphenyl)-1,2,4-oxadiazole, 3-(5-fluoropyrid-2-yl)-5-(3-cyano-5-methylphenyl)-1,2,4-oxadiazole, 3-(5-cyanopyrid-2-yl)-5-(3-cyano-5-thiomethylphenyl)-1,2,4-oxadiazole, 3-(5-fluoropyrid-2-yl)-5-(4-cyano-2-thienyl)-1,2,4-oxadiazole, 3-(2-pyridyl)-5-(3-cyano-5-trifluoromethylphenyl)-1,2,4-oxadiazole, 3-(5-fluoro-2-pyridyl)-5-(3-cyano-5-trifluoromethylphenyl)-1,2,4-oxadiazole, 3-(5-fluoro-pyrid-2-yl)-5-[3-(3-pyridyl)phenyl]-1,2,4-oxadiazole, 5-[3-methyl-5-(3-pyridyl)-pyrid-4-yl]-3-(2-pyridyl)-1,2,4-oxadiazole, 5-[3-methoxy-5-(3-pyridyl)-pyrid-4-yl]-3-(2-pyridyl)-1,2,4-oxadiazole, 5-(2-pyridyl)-3-[5-(3-pyridyl)-pyrid-3-yl]-1,2,4-oxadiazole, 5-(2-pyridyl)-3-[3-(3-pyridyl)-phenyl]-1,2,4-oxadiazole, 5-(5-fluoro-pyrid-2-yl)-3-[3-fluoro-5-(3-pyridyl)-phenyl]-1,2,4-oxadiazole, 3-(2-pyridyl)-5-(3-carboxy-5-methoxyphenyl)-1,2,4-oxadiazole, 3-(2-pyridyl)-5-(3-(carboxamido)-5-methoxyphenyl)-1,2,4-oxadiazole, 3-(2-pyridyl)-5-(3-cyano-5-(2-N,N-dimethylaminoethoxy)phenyl)-1,2,4-oxadiazole, 3-(2-pyridyl)-5-(3-cyano-5-(N,N-dimethylaminopropoxy)phenyl)-1,2,4-oxadiazole, 3-(2-pyridyl)-5-(3-cyano-5-(2-aminoethoxy)phenyl)-1,2,4-oxadiazole, 3-(2-pyridyl)-5-(3-cyano-5-(2-hydroxyethoxy)phenyl)-1,2,4-oxadiazole, 3-(2-pyridyl)-5-(3-cyano-5-isopropoxyphenyl)-1,2,4-oxadiazole, 3-(2-pyridyl)-5-(3-cyano-5-ethoxyphenyl)-1,2,4-oxadiazole, 3-(2-pyridyl)-5-(3-cyano-5-(2,2,2-trifluoroethoxy)phenyl)-1,2,4-oxadiazole, 3-(2-pyridyl)-5-(3-cyano-5-cyclopropylmethoxyphenyl)-1,2,4-oxadiazole, 3-(2-pyridyl)-5-(3-amino-5-cyanophenyl)-1,2,4-oxadiazole, 3-(5-fluoropyrid-2-yl)-5-(3-amino-5-cyanophenyl)-1,2,4-oxadiazole, 3-(2-pyridyl)-5-(3-cyano-5-(trifluoromethylsulfonyloxy)phenyl)-1,2,4-oxadiazole, 3-(2-pyridyl)-5-(3-cyano-5-(2-methoxy-2-oxoethoxy)phenyl)-1,2,4-oxadiazole, 3-(2-pyridyl)-5-(3-cyano-5-(2-tert-butoxy-2-oxoethoxy)phenyl)-1,2,4-oxadiazole, 3-(2-pyridyl)-5-(3-cyano-5-(methoxymethoxy)phenyl)-1,2,4-oxadiazole, 3-(2-pyridyl)-5-(3-cyano-5-(methoxyethoxy)phenyl)-1,2,4-oxadiazole, 3-(5-fluoropyrid-2-yl)-5-(3-cyano-5-cyclopentylaminophenyl)-1,2,4-oxadiazole, 3-(2-pyridyl)-5-(3-cyano-5-hexyloxyphenyl)-1,2,4-oxadiazole, 3-(2-pyridyl)-5-(3-cyano-5-(dimethylamino)carbonylphenyl)-1,2,4-oxadiazole, 3-(5-fluoropyrid-2-yl)-5-(3-cyano-5-ethylaminophenyl)-1,2,4-oxadiazole, 3-(5-fluoropyrid-2-yl)-5-(3-cyano-5-diethylaminophenyl)-1,2,4-oxadiazole, 3-(5-fluoro-pyrid-2-yl)-5-[3-fluoro-5-(1H-tetraazol-5-yl)-phenyl]-1,2,4-oxadiazole, 5-[3-fluoro-5-(1-methyl-1H-tetraazol-5-yl)-phenyl]-3-(5-fluoro-pyrid-2-yl)-1,2,4-oxadiazole, 3-(5-fluoro-2-pyridyl)-5-(3-(1-benzyl-1,2,5,6-tetrahydropyridin-3-yl)-5-fluorophenyl)-1,2,4-oxadiazole, 3-(5-fluoro-2-pyridyl)-5-(3-fluoro-5-(1H-imidazol-4-yl)phenyl)-1,2,4-oxadiazole, 1-(3-cyanophenyl)-4-(5-fluoro-2-pyridyl)-1H-imidazole, 3-(2-pyridyl)-5-(3-(1H-imidazol-1-yl)-5-thiomethoxyphenyl)-1,2,4-oxadiazole, 3-(3-cyano-5-(1H-imidazol-1-yl)phenyl)-5-(2-pyridyl)-1,2,4-oxadiazole, 3-(2-pyridyl)-5-(3-cyano-5-(1H-imidazol-1-yl)phenyl)-1,2,4-oxadiazole, 2-(3-iodophenyl)-4-(pyridin-2-yl)-1,3-thiazole, 3-(2-pyridyl)-2-(3-cyanophenyl)-furan, 3-(5-fluoro-2-pyridyl)-2-(3-cyanophenyl)-furan, 3-(5-chloro-2-pyridyl)-2-(3-cyanophenyl)-furan, 2-(2-pyridyl)-5-(5-fluoro-3-(1-imidazolyl)phenyl)-furan, 3-(5-(2-pyridyl)-2-furyl)-benzonitrile, 3-(5-(5-chloro-2-pyridyl)-2-furyl)-benzonitrile, 3-(5-(5-cyano-2-pyridyl)-2-furyl)-benzonitrile, 3-(5-(5-fluoro-2-pyridyl)-2-furyl)-benzonitrile, 3-fluoro-5-(5-(2-pyridyl)-2-furyl)-benzonitrile, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyano-5-fluorophenyl)-4-(2-pyridyl)-1,3-oxazole, 2-(5-allyloxy-3-cyanophenyl)-4-(5-fluoropyrid-2-yl)-1,3-oxazole, 2-(3-cyano-5-methoxyphenyl)-4-(pyrid-2-yl)-1,3-oxazole, 2-(3-cyano-5-methoxyphenyl)-4-(5-fluoropyrid-2-yl)-1,3-oxazole, 2-(3-cyano-5-n-propyloxyphenyl)-4-(5-fluoropyrid-2-yl)-1,3-oxazole, and 2-(3-cyano-5-methoxyphenyl)-4-(4-pyrid-2-yl)-5-chloro-1,3-oxazole.

12. A method according to claim 1, wherein said cough is chronic cough.

13. A method according to claim 1, wherein said cough is acute cough.

14. A method according to claim 4, wherein Ar¹ is substituted by one or more moieties selected from the group consisting of CF₃, F and Cl.

15. A method according to claim 2, wherein said cough is chronic cough.

16. A method according to claim 3, wherein said cough is chronic cough.

17. A method according to claim 4, wherein said cough is chronic cough.

18. A method according to claim 5, wherein said cough is chronic cough.

19. A method according to claim 6, wherein said cough is chronic cough.

20. A method according to claim 7, wherein said cough is chronic cough.
